(19) United States
(12) Patent Application Publication
(10) Pub. No.: US 2009/0192473 A1
(43) Pub. Date: Jul. 30, 2009

(54) SYSTEMS FOR CONTROLLED CLOSURE OF BODY LUMENS

(75) Inventors: Michael D. CROCKER, Half Moon Bay, CA (US); Paul MUNOZ, Laguna Beach, CA (US)

Correspondence Address: LEVINE BAGADE HAN LLP, 2400 GENG ROAD, SUITE 120, PALO ALTO, CA, 94303, US

(73) Assignee: ArterioPort, Inc., Menlo Park, CA (US)

(21) Appl. No.: 12/019,036

(22) Filed: Jan. 24, 2008

(51) Int. Cl. A61M 25/01

(52) U.S. Cl. 604/264

(57) ABSTRACT

Systems for controlled closure of body lumens may utilize a single access port which is attached to a vessel wall and allows for controlled closure and/or insertion of small to large sized instruments and catheters. The vascular access device generally includes a first frame and a second frame having an access port secured between the respective frames, e.g. via adhesive, welding, etc. Each respective frame may be configured to include a support member which may be shaped in a variety of configurations so long as an access opening is defined by the support member. A number of retaining members may extend radially from its respective support member and each retaining member may be configured so as to compress or “sandwich” the vessel tissue which surrounds the tissue opening between the retaining members on the inner and outer surfaces of tissue to securely position the device along the vessel wall.

### FIELD OF THE INVENTION

The present invention relates to devices and methods for accessing and/or controlling vascular access puncture sites. More particularly, the present invention relates to devices and methods for sealing, accessing, and/or controlling entry through vascular puncture sites via self-adjusting entry devices.

### BACKGROUND OF THE INVENTION

The increasing success of interventional techniques to access and repair structural disorders of the heart and vascular system has led to increasing demand for such procedures. Methods to deploy either intra-vascular stents or valve repair devices generally utilize the insertion of catheters through arteries and veins in the upper or lower extremities. As the technology and the ability to treat a wider-range of medical conditions evolve, the devices delivered have increased in size. Accordingly, closure of larger sized holes left by larger diameter catheters may be problematic for a patient.

A common cause of patient morbidity for interventional techniques is vascular access site complications, such as hematomas, pseudoaneurysms, and retroperitoneal bleeding. Such complications are likely to increase in frequency and severity with anti-coagulation and the use of larger diameter catheters used to deliver the endovascular devices. These complications may lead to prolonged hospital stay, increased costs, and the possible need for transfusion or surgery. Additionally, complications may lead to patient dissatisfaction and discomfort.

Manual compression of a vascular access site is typically utilized to achieve hemostasis of the opening when the size of the catheter sheath used is 6 F or less. But endovascular treatment of larger aneurysms and valvular diseases in an anti-coagulated patient generally require catheter sheaths in the range of 18-24 F. Although a cut-down can be performed by a vascular surgeon to directly close the access site in the artery or vein, alternative and less invasive methods are desirable. Furthermore, dilation of the artery or vein by the increasing diameter catheters can lead to damage and tearing of the vessel wall, making them less amenable to direct closure.

Conventional methods and devices used to close vessel puncture sites or ports, typically in the 6-8 F range, generally fall into the following categories: direct pressure, sealant-based devices, suture-based devices, staple-based devices, and direct closure by cut-down and vascular suture. However, each of these methods and devices has their limitations. For instance, most of these methods and devices have failure rates of up to 30% when utilized on relatively large diameter holes, e.g., 18 F or greater. Moreover, suture or staple-mediated devices also have the disadvantage of potentially narrowing the artery caliber and thus are contraindicated for use in relatively small vessels, e.g., 5 mm or less.

Additionally, procedures requiring repeated access to vessels may require the creation or multiple access sites is closure by many conventional devices and methods fail to allow for repeated access through the same site.

Accordingly, there is a need for methods and devices which allow for the controlled access by any number of various sized devices to any number of various diameter vessels while maintaining hemostasis as well as for allowing repeated access to a vessel through a single access site as necessary or desirable.

### SUMMARY OF THE INVENTION

Access and/or closure ports and methods of use for controlling access to vascular bodies may allow for a single access port which is adhered, connected, or otherwise attached to the wall of a hollow body lumen, e.g., vessels (arteries, veins, etc.), organs (bladder, stomach, etc.), etc., and allows for, but is not limited to, control of small to large sized vascular defects, use with anticoagulation agents, rapid sheath removal, early ambulation of the patient, access through the same port, maintaining a size of the vessel lumen after repair, etc. Moreover, such an access and/or closure port may allow a user to access and/or re-access the same hollow body lumen, such as an artery and/or vein, of patients utilizing various diameter catheters and instruments. Although examples of use of the device may be described herein in relation to vessels, it is to be understood that the devices and methods may also be utilized with other hollow body lumens such as the bladder, stomach, etc.

When an instrument or catheter is inserted through the flaps of such an access port, the flaps may be pushed inwardly into the vessel lumen to provide a channel for passage of the instrument or catheter sheath while the access port shields the vessel wall from damage. The outer periphery of the access port may remain intact and the flaps may allow the insertion of various sized catheter sheaths. Removal of the instrument or catheter may allow for the return of the patch flaps to a neutral position. The access port would allow re-access of the vessel, it necessary, even in the anti-coagulated patient.

Generally, the vascular closure device may comprise a first frame and a second frame having an access port secured between the respective frames, e.g. via adhesive, welding, etc. Each respective frame may be configured to include a support member which may be shaped in a variety of configurations, e.g., circular, elliptical, rectangular, triangular, etc. so long as an access opening is defined by the support member. A number of retaining members may extend radially from the respective support member and each retaining member may be configured so as to have an atraumatic form or shape to prevent injury to the surrounding, tissues.

Each retaining member may itself form a closed-loop structure to define an opening therethrough. Each support member may be integral with the retaining members to form a unitary structure although separate retaining members may also be connected or coupled to each respective support member in other variations. In either case, the frames may be fabricated from a variety of biocompatible materials, e.g., stainless steel, shape memory, alloys such as Nitinol, polymeric materials, etc. The access port may be secured between the frame members such that the access port forms the valved or flapped entry. The access port may be made from any variety of flexible biocompatible materials such as ePTFE, other fluoropolymers, polymers, polymeric blends thereof, elastomers, etc.

The frames may also be made from a bioabsorbable material such that they degrade or become absorbed into the patient body over a period of time. In such a variation, the device may be utilized to appose the edges of the tissue opening against one another such that healing of the tissue and closure of the opening is achieved with the eventual degradation and/or absorption of the device.

Alternatively, rather than having a port which is accessible or re-accessible therethrough, the port may be closed or sealed entirely in which case the closed-loop structure forms an integral device which is solid rather than defining an opening. The area defined by the frame may be comprised of the same material and/or integral with the frame or it may utilize any one of the materials to cover the area to provide for a complete integral seal. In this example, the device may be utilized to completely seal the opening to the hollow body lumen, e.g., after completion of a procedure, rather than providing for an entry or re-entry pathway in the body lumen. Accordingly, the device may be utilized for various purposes, e.g., access to a body lumen, closure or sealing of an opening to the body lumen, and/or re-access to a body lumen after placement of the device along the body lumen, or any combination thereof.

The adjoining frames may be secured to one another directly via welding or vial other securement mechanisms such as clips, screws, adhesives, etc. to compress or secure the periphery of the access port between the support member. Additionally, the retaining members from each respective frame may be positioned such that they define an alternating pattern. Alternatively, the retaining members may be aligned with respect to one another.

When deployed, the vascular access or entry device may have its radially extending retaining members retracted in a low-profile delivery configuration such that they extend radially when free from the constraints of the delivery instrument to compress or “sandwich” the tissue therebetween which surrounds the tissue opening to securely position the device along the vessel % all. To accommodate any curvature of the vessel wall, the frames may be configured to be suitably pliable to conform to any curved tissue when extended. Alternatively, the frames may be preformed to have a range of curvatures over a plane of the device such that the device may form a curved access or entry port which conforms to the natural curvature of the vessel.

### BRIEF DESCRIPTION OF THE DRAWINGS

FIGS. 1A and 1B illustrate perspective exploded and assembly views, respectively, of a variation of a vascular closure and/or entry device having an access port secured between a first and second frame.

FIGS. 2A and 2B illustrate perspective exploded & assembly views, respectively, of the assembly of FIGS. 1A and 1B with the radially extending retaining members retracted in a low-profile delivery configuration.

FIGS. 3A and 3B illustrate partial cross-sectional perspective views of a needle and dilator, respectively, introduced into a vessel for placement of a vascular entry device.

FIGS. 4A and 4B illustrate partial cross-sectional perspective views of a delivery instrument introduced at least partially into the vessel for deploying a vascular entry device.

FIGS. 5A and 5B illustrate side and perspective views, respectively, of a vascular entry device deployed along the vessel wall with the radially extending retaining members placed into apposition along the inner and outer surfaces of the vessel wall.

FIGS. 6A to 6C illustrate top and perspective views, respectively, of a vascular entry device placed along the vessel wall while maintaining hemostasis via the access port.

FIGS. 6D and 6E illustrate examples of various diameter instruments which may be inserted through the vascular entry device during the same procedure and/or between different procedures through the same device while maintaining hemostasis though the access port.

FIGS. 7A and 7B illustrate top and perspective views of another variation of a vascular device frame showing the radially extending retaining members uniformly positioned along the support member.

FIG. 8 illustrates a top view of frame members overlaid upon one another and the relative positioning of their retaining members in an alternating pattern.

FIG. 9A illustrates a top view of another variation of a frame member having an integrated scaffold for several flap or valve members.

FIGS. 9B and 9C show detail views of the intersection, of the flap or valve scaffold and the connection between the scaffold and support member, respectively.

FIG. 9D shows a perspective view of the frame member of FIG. 9A.

FIG. 10A illustrates a partial cross-sectional side view of another variation of the vascular entry device having a connecting portion between frame members for vessel walls having a relatively greater thickness.

FIGS. 10B and 10C illustrate perspective views of the device of FIG. 10A along the vessel wall.

FIG. 11A illustrates another variation of the access port comprised of multiple overlapping leaflets or formed loops.

FIG. 11B illustrates a top view of a vascular entry device having the multiple leaflets or formed loops overlapping with adjacent leaflets to form an access port.

### DETAILED DESCRIPTION OF THE INVENTION

Vascular access and/or sealing control devices and methods of use may allow for a single access port which is adhered, connected, or otherwise attached to the wall of a hollow body lumen. e.g., vessels (arteries, veins, etc.), organs (bladder, stomach, etc.), etc., and allows for, but is not limited to, control of small to large sized openings such as vascular defects (e.g., large sized vascular defects or openings may range anywhere from 12 F to 24 F or greater), use with anticoagulation agents, rapid sheath removal, early ambulation of the patient, access through the same port, maintaining a size of the vessel lumen after repair, etc. Although examples of use of the device may be described herein in relation to vessels, it is to be understood that the devices and methods may also be utilized with other hollow body lumens such as the bladder, stomach, etc.

Moreover, such an access port may allow a user to access and/or re-access the same artery and/or vein of patients utilizing various diameter catheters and instruments. For instance, patients who may require long-term indwelling catheters or those who require repeated intravascular access, e.g., hemodialysis patients, may benefit. Thus, after a procedure utilizing the access port, the tissue region surrounding the access port may be closed upon the port and left implanted in the patient as the access port provides hemostasis of the vessel. If re-entry or further access is desired to the vessel for any further procedures, the access port may again be entered through the tissue region for the re-entry or re-introduction of one or more instruments or catheters through the same port without having to create ally additional entry paths into the vessel lumen. The re-entry or further access through the access port may be obtained intra-procedurally or post-procedurally spanning anywhere from hours, days, weeks, months, or even years from an initial procedure. Moreover, the access port may be left implanted within the patient permanently, if so desired, to provide this re-entry path into the vessel for future use or it may be optionally removed at any time and the opening through the vessel may be closed surgically.

Examples of such devices are described in greater detail in U.S. Pat. App. 11/864,446 filed Sep. 28, 2007, which is incorporated herein by reference in its entirety. Generally, a vessel entry assembly may allow a user to create a controlled vascular opening by maintaining a vascular puncture while protecting the vessel during insertion and/or withdrawal of relatively large diameter sheaths, catheters, or instruments. A vascular access or entry assembly may be deployed within or against the vessel wall where the access port may allow for the automatic closure and sealing of a vascular opening to maintain hemostasis when instruments or catheters are withdrawn from the device.

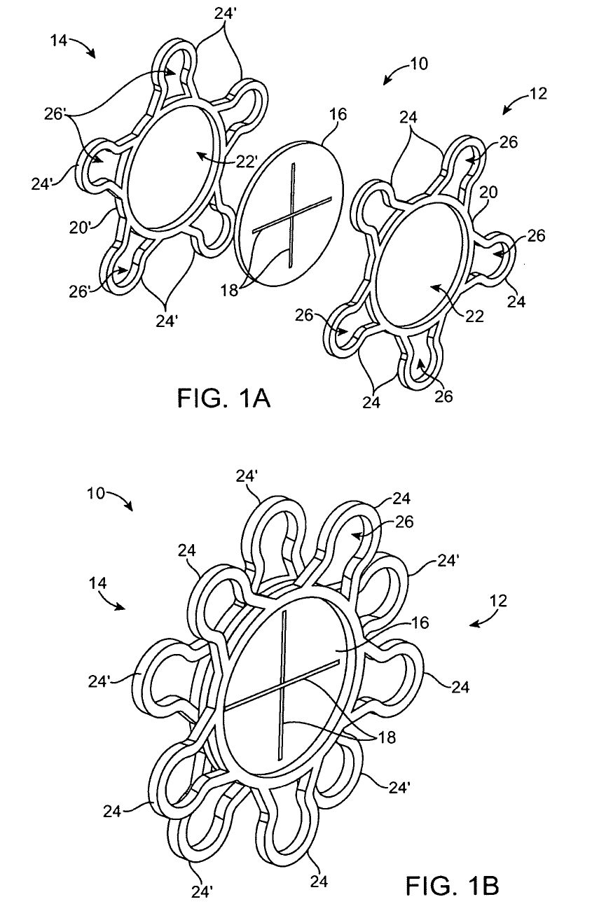

Turning now to FIGS. 1A and 1B, an example of a variation of a vascular entry device is illustrated in exploded and assembly perspective views, respectively. In this particular variation, vascular entry assembly 10 may be comprised generally of a first frame 12 and a second frame 14 having access port 16 secured between the respective frames 12, 14. e.g., via adhesive, welding, etc. Each respective frame 12, 14 may be configured to include a support member 20, 20′ having a planar shape and which may be shaped in a variety of configurations, e.g., circular as shown, elliptical rectangular, triangular, etc. so long, is an access opening 22, 22′ is defined by support member 20, 20′. A number of retaining, members 24, 24′ may extend radially from the respective support member 20, 20′ and each retaining member 24, 24′ may be configured so as to have an atraumatic form or shape to prevent injury to the surrounding tissues. Although the example illustrates five retaining members 24, 24′ uniformly spaced apart and extending radially relative to one another, fewer than five members or greater than five members may be utilized if desired.

Each retaining member 24, 24′ may itself form a closed-loop structure to define an opening 26, 26′ therethrough. The variation illustrated shows retaining members 24, 24′ defining an arcuate portion along its contour although other shapes and contours may be utilized so long as the shape presents an atraumatic surface to the contacted tissue. Each support member 20, 20′ may be integral with retaining members 24, 24′ to form a unitary structure although separate retaining members 24, 24′ may also be connected or coupled to each respective support member 20, 20′ in other variations. In either case, frames 12, 14 may be fabricated from a variety of biocompatible materials, e.g., stainless steel, shape memory alloys such as Nitinol, polymeric materials, etc.

The frames 12, 14 may also be made from a bioabsorbable material such that they degrade or become absorbed into the patient body over a period of time. In such a variation, the device may be utilized to appose the edges of the tissue opening against one another such that healing of the tissue and closure of the opening is achieved with the eventual degradation and/or absorption of the device.

Alternatively, rather than having a port which is accessible or re-accessible therethrough, the port may be closed or sealed entirely in which case the closed-loop structure forms an integral device which is solid rather than defining an opening. The area defined by the frame may be comprised of the same material and/or integral with the frame or it may utilize any one of the materials to cover the area to provide for a complete integral seal. In this example, the device may be utilized to completely seal the opening to the hollow body lumen, e.g., after completion of a procedure, rather than providing for an entry or re-entry pathway in the body lumen. Accordingly, the device may be utilized for various purposes, e.g., access to a body lumen, closure or sealing of an opening to the body lumen, and/or re-access to a body lumen after placement of the device along the body lumen, or any combination thereof.

Access port 16 may be secured between the frame members 12, 14 such that access port 16 forms the valved or flapped entry over access openings 22, 22′. Moreover, access port 16 may define one or more partitions or seams 18, e.g., one partition or two transversely defined relative to one another as shown, over access port 16 to form the valved or flapped opening through which one or more instruments may be, inserted through. Access port 1.6 may be made from any variety of flexible biocompatible materials such as ePTFE, other fluoropolymers, polymers, polymeric blends thereof, elastomers, etc. and access port 16 may also optionally incorporate expandable biomaterials along the seams 18 to allow for swelling and expansion and sealing of the seams 18 when placed in contact with blood or when temperature is increased. Additionally in other alternatives, access port 16 and/or frames 12, 14 may also incorporate drug-eluting agents to facilitate the healing of the acute wound site. Once vascular entry device 10 has been desirably placed within or along the vessel and one or more procedures through access port 1.6 has been accomplished, access port 16 may be simply left or partitions 18 may be securely tightened, e.g., via adhesives or sutures.

The perspective view of FIG. 1B illustrates access port 16 positioned between frame members 12, 14. The adjoining frames 12, 14 may be secured to one another directly via welding or via other securement mechanisms such as clips, screws, adhesives, etc. to compress or secure the periphery of access port 16 between support member 20, 20′. Additionally, retaining members 24, 24′ from each respective frame 12, 14 may be positioned such that they define an alternating pattern, as illustrated. Alternatively, retaining members 24, 24′ may be aligned with respect to one another.

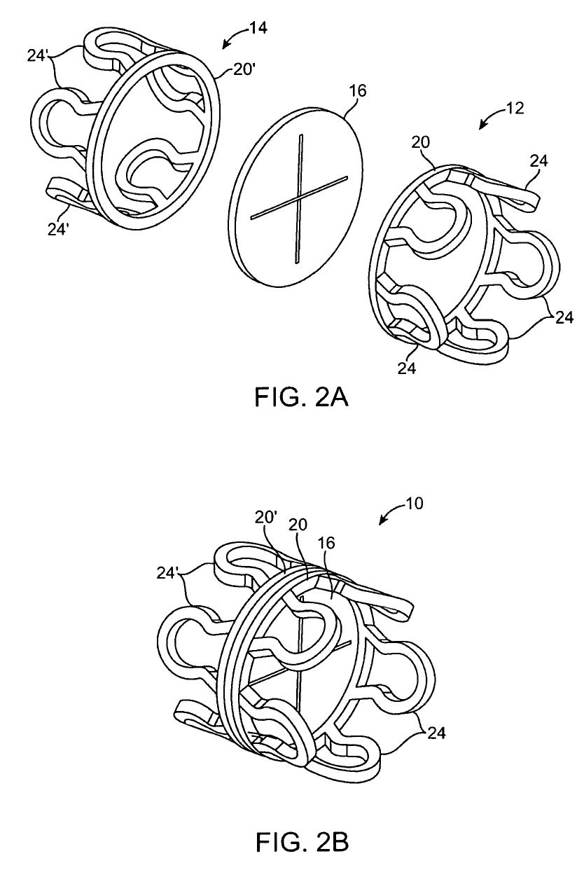

When deployed, vascular access or entry device 10 may have its radially extending retaining members 24, 24′ retracted in a low-profile delivery configuration, as illustrated in the perspective exploded & assembly views of FIGS. 2A and 2B, respectively. In this variation, the low-profile configuration may involve having retaining members 24, 24′ constrained from its radially extending configuration into a configuration where retaining members 24, 24′ are positioned transversely relative to access port 16 to facilitate deliver-y and placement of the vascular entry device, as described below.

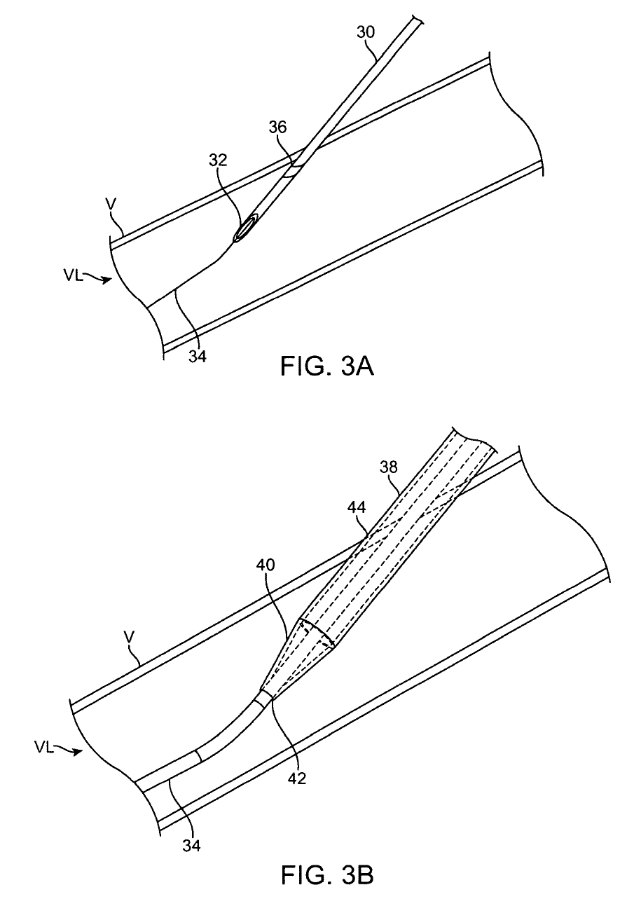

In deploying and placing the vascular entry device, the targeted vessel V along which the device is to be placed may be pierced via needle 30 to create an initial vessel opening 36, as illustrated in the partial cross-sectional perspective view of FIG. 3A. With needle 30 advanced at least partially into the vessel lumen VL, an optional guidewire 34 may be advanced through needle opening 32 for placement at least partially within the vessel lumen VL. With guidewire 34 positioned through initial vessel opening 36 and within the vessel lumen VL, needle 30 may be removed and a separate dilating instrument 38 having a tapered dilating tip 40 may be advanced over guidewire 34 through dilator lumen 42 and into the initial vessel opening 36 to widen the opening into a dilated opening 44, as shown in the perspective view of FIG. 3B. This technique is similar to the Seldinger technique and may be optionally utilized with visualization modalities to facilitate the initial entry into the vessel V, e.g., fluoroscopy, ultrasound, etc., if so desired.

In other variations, access to the targeted vessel V may be obtained by cutting down through the surrounding skin and accessing the vessel. An opening may then be cut directly along the vessel wall to form the initial entry into the vessel lumen VL. In yet another alternative, after dilation of the opening or in place of an initial dilation, a tissue punch instrument may be inserted into the initial opening and used to remove tissue surrounding the initial opening to create a larger diameter opening for access into the vessel lumen VL.

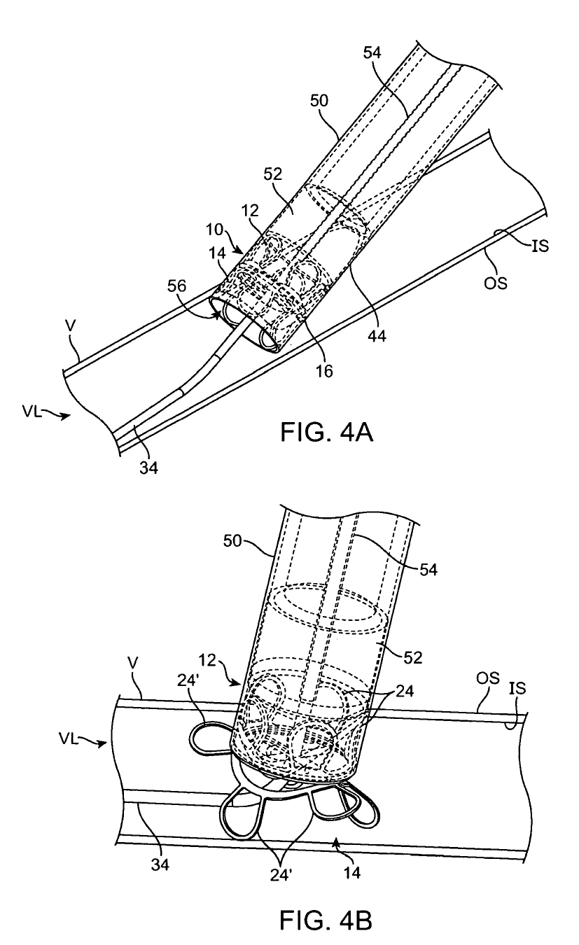

With the tissue opening 44 dilated or otherwise widened, the dilating instrument 38 (if used) may be removed from guidewire 34 and a separate delivery instrument 50 may be advanced along guidewire 34 and inserted at least partially into tissue opening 44, as illustrated in the perspective view of FIG. 4A. Delivery instrument 50 may generally comprise an instrument body defining an instrument lumen 56 and a plunger 52 slidably positioned within. Plunger 52 may be advanced or retracted via plunger shaft 54, through which guidewire 34 may be positioned. Vascular access device 10 may be positioned within instrument lumen 56 such that its radially extending members 24, 24′ are in its low-profile configuration while constrained via delivery instrument 50, as shown.

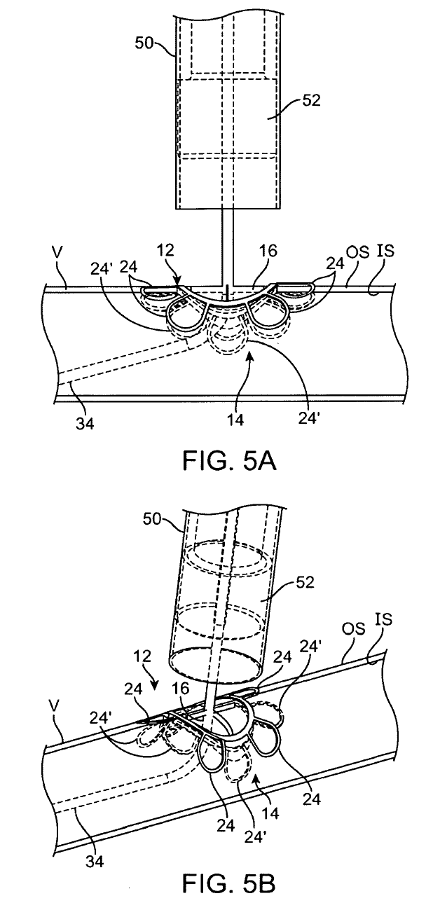

With second frame 14 positioned distally of first frame 12 within instrument lumen 56 and with the distal opening of delivery instrument 50 inserted at least partially into tissue opening 44, plunger 52 may be pushed distally via plunger shaft 54 to abut against first frame 12 such that device 10 is urged distally through instrument lumen 56. Device 1.0 may be urged distally until the constrained retaining members 24′ of second frame 14 are free to extend radially within the vessel lumen VL and into contact against the inner surface of the vessel V, as shown in FIG. 4B. With the retaining members 24′ of second frame 14 expanded, the constrained retaining members 24 of first frame 12 may then be pushed by plunger 52 out of instrument lumen 56 to allow for the extension of the retaining, members 24 into their radial configuration and into contact against the outer surface OS of the vessel V, as shown in the side and perspective views of FIGS. 5A and 5B. The radially extended members of both first and second frames 12, 14 along the respective outer and inner surfaces OS, IS of the vessel V may thus compress or “sandwich” the tissue therebetween which surrounds the tissue opening to securely position the device 10 along the vessel wall. The alternating pattern between the retaining members 24, 24′ may also facilitate the securement of device 10 relative to the tissue while maintaining hemostasis to prevent or inhibit any leakage of blood from vessel lumen VL.

To accommodate any curvature of the vessel wall, the frames 12, 14 may be configured to be suitably pliable to conform to any curved tissue when extended. Alternatively, frames 12, 14 may be preformed to have a range of curvatures over a plane of the device 10 such that when retaining members 24, 24′ and support members 20, 20′ are freed from the constraints of delivery instrument 50, device 10 may form a curved access or entry port which conforms to the natural curvature of the vessel V, as illustrated in FIGS. 5A and 5B. This may further prevent or reduce any trauma between the device and the surrounding vessel tissue. Moreover, the preformed curvature of the device 10 may be varied depending upon the vessel which the device 10 is to be placed. Accordingly, device 10 may be provided in a range of different curvatures or it may be configured to have an initial curvature which may flex to conform to the contours of a particular vessel.

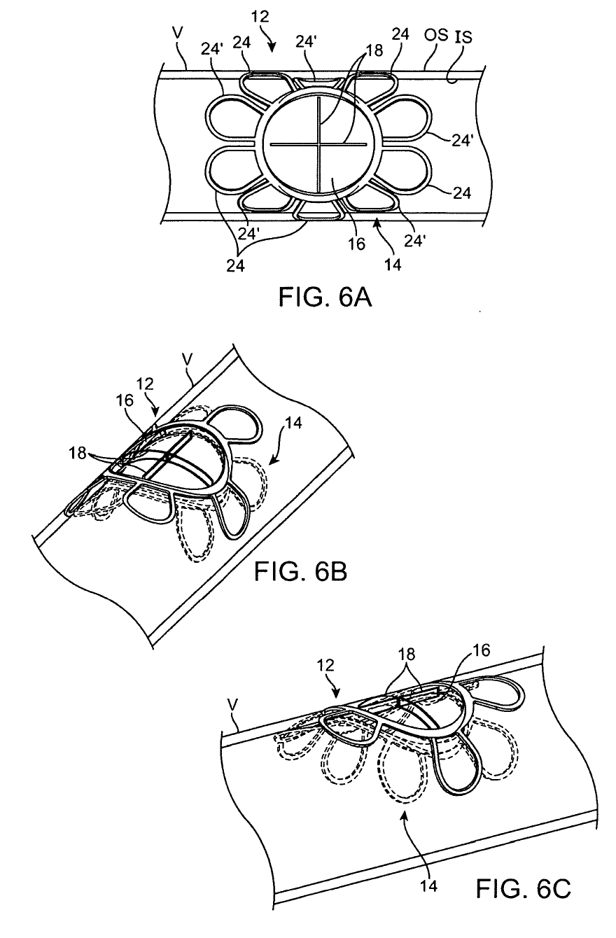

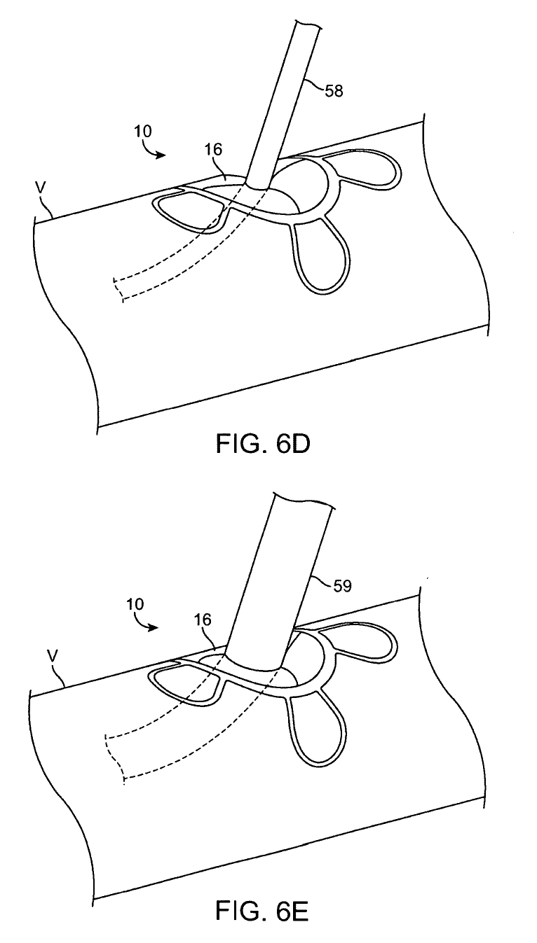

With the retaining members 24, 24′ secured against or along the vessel outer and inner surfaces OS, IS, guidewire 34 may be withdrawn from vessel lumen VL and from access port 16 to allow the valve or flaps of access port 16 to fully close and maintain hemostasis such that blood or fluids are prevented from leaking through the seams 18 of access port 16, as illustrated in the top view of FIG. 6A and perspective views of FIGS. 6B and 6C. Alternatively, guidewire 34 may be left in place passing through access port 16 and into the vessel lumen VL for use with other instruments. In either case, once the device 10 has been secured against or along the vessel V, the device 10 may allow a user to access and/or re-access the same vessel V utilizing various diameter catheters and instruments intra-operatively or post-operatively through the same port 16 without having to create any additional entry paths into the vessel lumen VL. For instance, FIG. 6D illustrates a perspective view of a first instrument 58 having a first diameter, e.g., 8 F, introduced into the vessel lumen VL for a procedure. Intra-operatively or post-operatively, a second instrument 59 having a second diameter, e.g., 14 F as illustrated in FIG. 6E, may be introduced into the same vessel lumen VL through the same access port 16 to further treat the patient. Withdrawal of the instruments allows for access port 16 to sufficiently seal to prevent any blood or fluid leakage therethrough. Thus, device 10 may be left in place within the patient, e.g., permanently if so desired, to allow the surrounding tissue to heal. Moreover, the flaps or valve of access port 16 may be optionally further sealed or closed via adhesives or sutures to ensure hemostasis. Alternatively, device 10 mats be removed after the one or more procedures are completed and the vessel V closed via adhesives, sutures, etc.

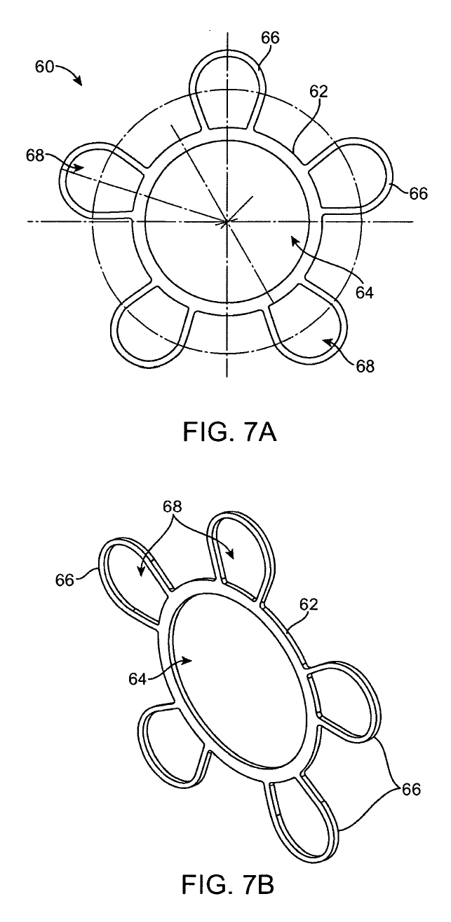

FIGS. 7A and 7B illustrate top and perspective views of a variation of a frame 60 halving radial retaining members 66 which are formed in a looped configuration, such as a leaflet or petal. Frame 60, as mentioned above, may be fabricated from a biocompatible material, e.g., stainless steel, shape memory alloy such as Nitinol, or a polymeric material, etc. to have a thickness of about, e.g., 0.006 in. The relatively small thickness of frame 60 allows for the frame, especially if positioned along the inner surface IS of the vessel lumen VL, to lie flush along the vessel surface to prevent thrombosis formation as well as to promote endothelialization of the frame against the tissue surface. Moreover, the smaller profile allows the unimpeded blood flow through the vessel lumen VL. The support member 62 defining access opening 64 may have a radial thickness of, e.g., 0.020 in. within an outer diameter of about 0.302 in. and inner diameter of about 0.262 in. Frame 60 may also be configured to be sized appropriately for placement upon the vessel. For instance, frame 60 may be sized to have an overall diameter of about 0.550 in. when accounting for the diameter of the retaining members 66.

The radial retaining members 66 may each define an opening 68 such that the retaining members 66 have a width of about, e.g., 0.010 in., and although five members are illustrated, fewer than five or more than five members may be utilized. If the five retaining members 66 are used, they may be uniformly positioned circumferentially around support member 62 at an angle of, e.g., 72′ relative to adjacent retaining members 66. Alternatively, retaining members 66 may be positioned along support member 62 at non-uniform angles for placement, for instance, along narrow vessels. These values relating to thickness, width, etc. for device 10 are merely intended to be illustrative and are given as examples of size considerations and are not intended to be limiting in any way of the scope of this disclosure.

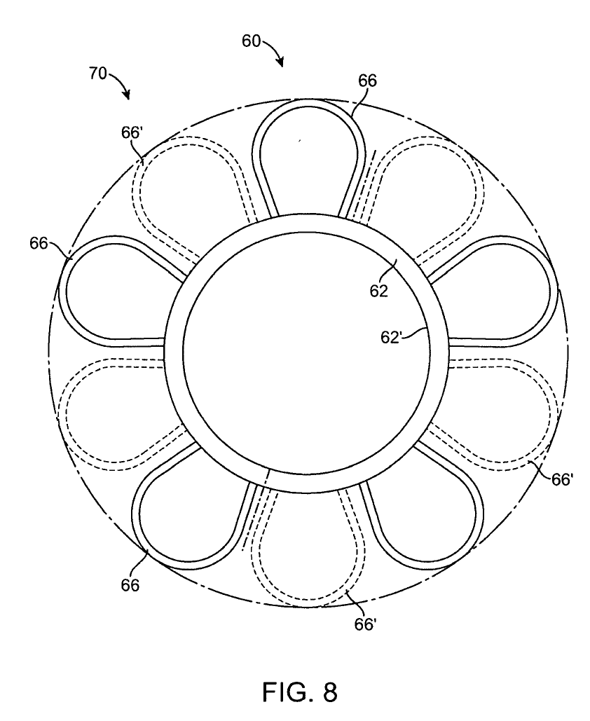

FIG. 8 illustrates a top view of frame 60 overlaid with second frame 70 (the access port has been omitted merely for clarity) to illustrate a method for the relative positioning between the frames 60, 70 of an entry device. As previously mentioned, the respective retaining members 66, 66′ of frames 60, 70 may be overlaid such that they are positioned in an alternating pattern to facilitate distribution of the stresses against the vessel walls and to enable a secure grip or hold of the retaining members 66, 66′ onto the vessel tissue. In other variations, rather than alternating the retaining members, they may be positioned such that they are aligned in apposition to grasp onto the tissue directly opposite to one another.

In yet another variation of the device of FIG. 8, rather than utilizing a first and second frame, a single support member may have a first set of retaining members configured for placement along the vessel inner surface IS and a second set of retaining members also extending from the same support member and configured for placement along the vessel outer surface OS. The retaining members may alternate in terms of placement along the inner or outer surface in the pattern as shown in FIG. 8. Accordingly, an access port may be attached directly to this single support member rather than being secured between two frames.

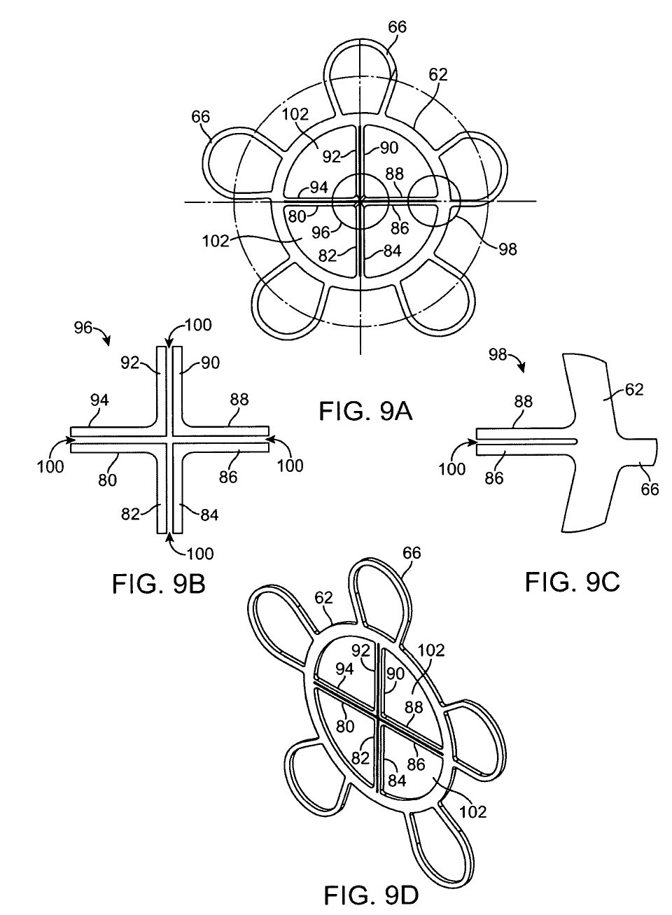

In another variation of the vascular access device, another configuration for the frame is illustrated in the top and perspective views of FIGS. 9A and 9D, respectively. In this example, a scaffold or frame may be integrated with or attached to the support member 62 to create a structural support for an integrated valve or flaps. Such an arrangement may be used alone with the apposing frame to create an overlapping series of flaps or it may be used in combination with a separate access port membrane positioned between the respective frames. In either case, the scaffold or frame may include pairs of valve support members each forming a leaflet or flap. For instance, valve support members 80, 82 and each respective pair of valve support members 84, 86, valve support members 88, 90, and valve support members 92, 94 may each form a leaflet or flap which is covered with a membrane 102, as described above. Moreover, each support member may have a thickness of about, e.g., 0.005 in., such that each formed leaflet or flap may independently flex and adjust to accommodate the variously-sized instruments passed through the opening to access the vessel lumen VL. Each formed leaflet or flap may define a channel or groove 100, e.g., having a width of about 0.003 in., between adjacent valve support members. For example, FIG. 9B illustrates a detailed view of intersection 96 where each respective pair of valve support members are adjacent or in proximity to one another with channels or grooves 100 separating the valve support members. As the frame members are overlaid atop one another to form the vessel access device, the channels or grooves 100 of each respective frame may overlap with the membrane of the opposing frame such that a sufficient seal is formed to prevent the leakage of blood therethrough. Alternatively, an additional polymeric material or membrane may overlap between adjacent leaflets or flaps to maintain hemostasis once implanted.

In yet another alternative, the valve support members may be formed on a single frame e.g., support member 62, to form the port. Thus, when the frame members are overlaid atop one another, the set of frame members on a single frame may form the port rather than having overlapped valves alone, adjacent frames.

FIG. 9C shows a detailed view of connection 98 illustrating adjacent valve support members 86, 88 extending integrally from support member 62 where the valve support members may be fabricated from a common material with the support member. Alternatively, the valve support members may be attached separately to support member 62 via any number of attachment mechanisms, e.g., welding or other mechanical attachment mechanisms. Although four respective leaflets or flaps are shown in the example, two leaflets or more than four leaflets may be utilized, as practicable.

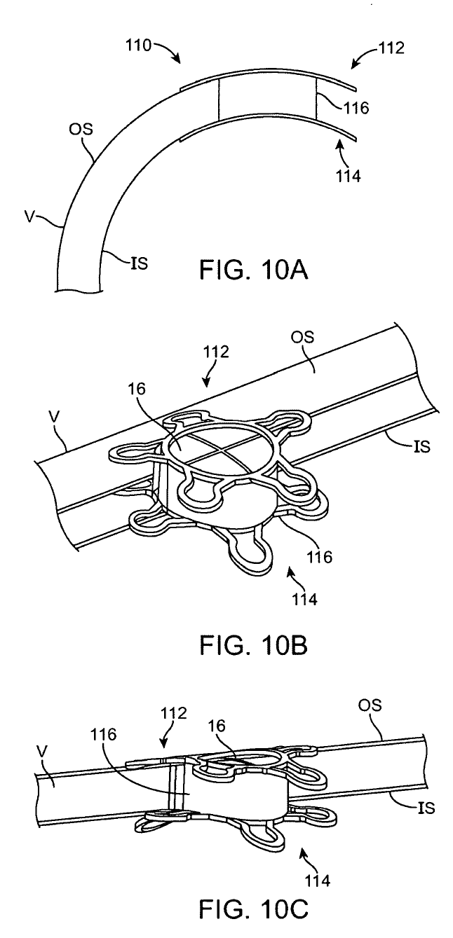

In yet another variation, FIG. 10A shows a partial cross-sectional side view of vascular entry assembly 110 which includes first frame 112 and second frame 114. In this example, rather than directly coupling or attaching the frames 112, 114 to one another or with an access port in-between, a connecting portion 116 having a height and defining a lumen therethrough may be attached to first and second frames 112, 114. Connecting portion 116 may be included to accommodate vessels having a relatively thick tissue wall. An access port 116 may be included and attached at either or both first and/or second frame 112, 114 or within connecting portion 116. FIGS. 10B and 10C illustrate various cross-sectional perspective views of assembly 110 showing the added height of connecting portion 116 facilitating placement and securement of assembly 110 within or along vessels V having a relatively larger thickness. The height of connecting portion 116 may be adjusted appropriately depending on the thickness of the target vessel V.

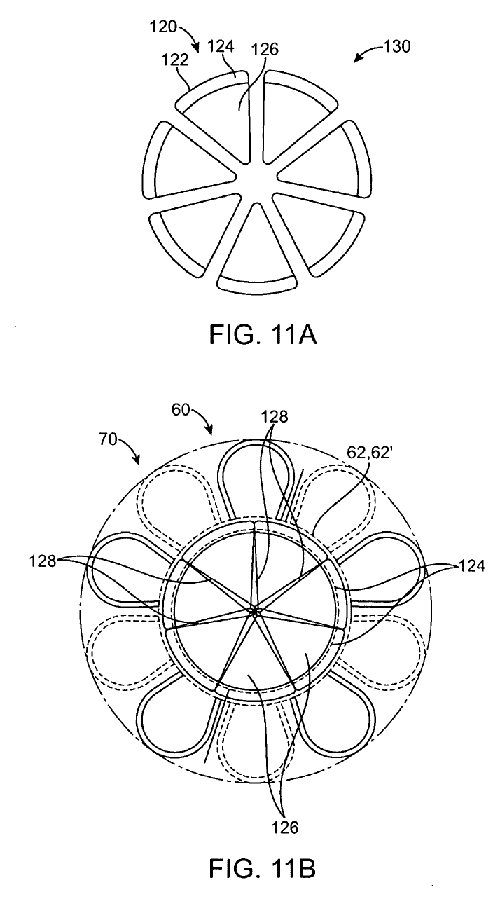

FIG. 11A illustrates another variation of the access port utilizing multiple overlapping leaflets or formed loops 130 where a biocompatible wire or ribbon 122, e.g., shape memory alloy such as Nitinol having a 0.003 in. thickness, may be formed into a leaflet or looped structure 120 having an angled or wedged shape. The angled portion may be coated or formed to have a fluid-impermeable membrane 126. e.g., by dipping into a polymeric solution such is polyurethane, formed at least partially over the looped structure 120. The multiple leaflets 130, once formed, may be positioned adjacent to one another in a circular pattern such that the uncovered portions 124 of each leaflet may be attached or connected, e.g., via adhesive or other mechanical mechanisms such as welding, to the support member 62, 62′ of first and/or second frames 60, 70. Each leaflet 120 may be formed such that the membrane 126 portion completely covers the access opening through frames 60, 70 and also such that each adjacent leaflet 120 overlaps at least partially with an adjacent leaflet 0.120 to create a leaflet overlap 128, as illustrated in the top view of FIG. 11B. When each adjacent leaflet 120 is closed, the overlapping portions 128 helps to maintain hemostasis and when one or more instruments are inserted through the device, the multiple leaflets 130 may easily conform to the instrument diameter to further help maintain hemostasis. Although seven leaflets are illustrated in this example, fewer or greater numbers of leaflets may be utilized which are accordingly sized.

The applications of the devices and methods discussed above are not limited to controlling, access to vessel lumens but may include other body lumens. Modification of the above-described assemblies and methods for carrying out the invention, combinations between different variations as practicable, and variations of aspects of the invention that are obvious to those of skill in the art are intended to be within the scope of the claims.

What is claimed is:

1. A closure device configured for placement along or upon a hollow body lumen to be sealed, comprising:
at least one support member having a thickness which is sized to lie flush along a surface of the hollow body lumen;
a first plurality of retaining members having a low-profile configuration and a deployed configuration, wherein the first plurality of retaining members are configured for placement against an inner surface of the hollow body lumen when deployed;
a second plurality of retaining members having a low-profile-configuration and a deployed configuration, wherein the second plurality of retaining members are configured for placement against an outer surface of the hollow body lumen when deployed such that the at least one support member is secured along or upon the hollow body lumen via the first and second plurality of retaining members; and
wherein the at least one support member defines an area sized to extend over an opening to be sealed along the hollow body lumen such that a fluid-impermeable seal is formed therebetween.

2. The device of claim 1 further comprising an access port positioned within or over an opening defined within the area, wherein the access port has one or more leaflets or flaps which are deformable into an open configuration and which are biased to reconfigure into a closed configuration which provides the fluid-impermeable seal.

3. The device of claim 1 wherein the at least one support member comprises a frame having a planar shape defining the opening therethrough.

4. The device of claim 1 wherein the at least one support member is comprised of a shape memory alloy.

5. The device of claim 1 wherein the at least one support member defines a curvature over a plane of the device.

6. The device of claim 1 wherein the first and second plurality of retaining members each define a looped shape extending radially from a circumference of the at least one support member.

7. The device of claim 6 wherein the first plurality of retaining members alternates with the second plurality of retaining members.

8. The device of claim 1 further comprising a second support member defining an area sized to extend over the opening to be sealed and having a thickness which is sized to lie flush along the surface of the hollow body lumen, wherein the at least one support member and the second support member are attached to one another.

9. The device of claim 8 wherein the first plurality of retaining members extends radially from the at least one support member and the second plurality of retaining members extends radially from the second support member.

10. The device of claim 8 wherein the access port is secured between the at least one support member and the second support member.

11. The device of claim 2 wherein the leaflets or flaps of the access port comprise a scaffold or frame connected to the at least one support member and extending over the opening.

12. The device of claim 2 wherein the leaflets or flaps each comprise a looped member having a fluid-impermeable portion extending over the opening such that adjacent leaflets or flaps overlap one another.

13. The device of claim 1 further comprising a connecting portion separating the first plurality and second plurality of retaining members from one another.

14. The device of claim 1 wherein the hollow body lumen comprises a vessel.

15. A closure device configured for placement along or upon a hollow body lumen to be accessed, comprising:
a first frame and a second frame attached to one another and each defining a common opening therethrough and having a thickness which is sized to lie flush along a surface of the hollow body lumen;
a first plurality of retaining members extending radially from the first frame and having a low-profile configuration and a deployed configuration, wherein the first plurality of retaining members are configured for placement against an inner surface of the hollow body lumen when deployed;
a second plurality of retaining members extending radially from the second frame having a low-profile configuration and a deployed configuration, wherein the second plurality of retaining members are configured for placement against an outer surface of the hollowed body lumen when deployed; and
an access port secured between the first and second frames within or over the opening, wherein the access port has one or more leaflets or flaps which are deformable into an open configuration and which are biased to reconfigure into a closed configuration which provides a fluid-impermeable seal.

16. The device of claim 15 wherein the first and second frames each have a planar shape.

17. The device of claim 15 wherein the first and second frames are comprised of a shape memory alloy.

18. The device of claim 15 wherein the first and second frames define a curvature over a plane of the device.

19. The device of claim 15 wherein the first plurality and second plurality of retaining members extend in an alternating pattern with respect to one another.

20. The device of claim 15 wherein the leaflets or flaps of the access port comprise a scaffold or frame extending over the opening.

21. The device of claim 15 wherein the leaflets or flaps each comprise a looped member having a fluid-impermeable portion extending over the opening such that adjacent leaflets or flaps overlap one another.

22. A method of sealing an opening to a hollow body lumens comprising:
advancing a first plurality of retaining members extending from a first frame and in a low-profile configuration through a passage along a wall of the hollow body lumen to be sealed;
radially extending the first plurality of retaining members into contact against an inner surface of the hollow body lumen; and
deploying a second plurality of retaining members extending from a second frame which is attached to the first frame into contact against an outer surface of the hollow body lumen such that a common opening defined through the first and second frames is positioned over the passage along the wall of the hollow body lumen,
wherein an access port is secured between the first and second frames within or over the opening such that one or more leaflets or flaps of the access port are deformable into an open configuration and are biased to reconfigure into a closed configuration which provides a fluid-impermeable seal through the opening.

23. The method of claim 22 wherein advancing comprises urging the first plurality of retaining members via a plunger from a delivery instrument through the passage.

24. The method of claim 22 wherein radially extending comprises positioning the first plurality of retaining members and the first frame flush along the inner surface of the vessel.

25. The method of claim 22 wherein deploying a second plurality of retaining members comprises con forming the first and second frames to a curvature of the hollow body lumen.

26. The method of claim 22 further comprising advancing at least a first instrument having a first diameter through the access port and into the vessel such that the leaflets or flaps conform to the first diameter.

27. The method of claim 26 further comprising advancing a second instrument having a second diameter through the access port and into the hollow body lumen such that the leaflets or flaps conform to the second diameter.

28. The method of claim 26 further comprising removing the first instrument such that the leaflets or flaps close to form the fluid-impermeable seal.

29. The method of claim 22 farther comprising sealing the leaflets or flaps to permanently obstruct the access port.
